(19) United States
(12) Patent Application Publication
(10) Pub. No.: US 2018/0228383 A1
(43) Pub. Date: Aug. 16, 2018

(54) BLOOD PRESSURE MEASURING DEVICE, BLOOD PRESSURE MEASUREMENT METHOD AND BLOOD PRESSURE MEASUREMENT PROGRAM

(71) Applicant: The University of Tokyo, Tokyo (JP)

(72) Inventors: Shinichi WARISAWA, Tokyo (JP); Rui FUKUI, Tokyo (JP); Haruyuki SANUKI, Tokyo (JP)

(21) Appl. No.: 15/897,442

(22) Filed: Feb. 15, 2018

(30) Foreign Application Priority Data
Feb. 15, 2017 (JP) 2017026090

(51) Int. Cl. A61B 5/021; A61B 5/0245; A61B 5/00; A61B 5/0456; A61B 5/024; G16H 40/63; G16H 50/20

(52) U.S. Cl. CPC A61B 5/02108; A61B 5/0245; A61B 5/7264; G16H 50/20; A61B 5/02416; G16H 40/63; A61B 5/0456

(57) ABSTRACT

Provided are a blood pressure measuring device, a blood pressure measurement method and a blood pressure measurement program that allow calculating blood pressure properly even for ambulatory users. A blood pressure measuring device 1 is provided with: an electrocardiogram acquisition unit 11 that acquires an electrocardiogram of a user; a pulse wave acquisition unit 12 that acquires a pulse wave of the user; a first extraction unit 13 that extracts a heart rate on the basis of the electrocardiogram; a second extraction unit 14 that extracts a pulse wave velocity on the basis of the electrocardiogram and the pulse wave; a third extraction unit 15 that extracts one or a plurality of feature values pertaining to the pulse wave, on the basis of the pulse wave; and a calculation unit 16 that calculates blood pressure of the user from the heart rate, the pulse wave velocity and the one or plurality of feature values extracted for the user, by a learner 17 that has learned, by nonparametric regression analysis, a relationship between blood pressure of each of a plurality of subjects and a heart rate, a pulse wave velocity and one or a plurality of feature values pertaining to a pulse wave extracted for each of the plurality of subjects.

### CROSS REFERENCE TO RELATED APPLICATIONS

The present application is based on Japanese Patent Application No. 2017-026090 filed on Feb. 15, 2017, the disclosures of which are incorporated herein by reference.

### BACKGROUND

### Field

The present invention relates to a blood pressure measuring device, a blood pressure measurement method and a blood pressure measurement program.

### Description of Related Art

Known conventional methods for measuring blood pressure include invasive methods, in which blood pressure is directly measured through insertion of a catheter into a blood vessel, and non-invasive methods. As the non-invasive methods, the Korotkoff method that involves wrapping a cuff around the arm or the like of a subject, applying pressure, and then measuring Korotkoff sounds is widely used. Known methods for measuring blood pressure without using a cuff include methods relying on pulse wave velocity, i.e. a propagation velocity at a time where a heart pulsation reaches a peripheral site.

Regarding a blood pressure measurement relying on pulse wave velocity, Patent Publication JP-A-2016-131825 discloses an information processing device that calculates pulse wave velocity as a feature value, and calculates a blood pressure value of a user on the basis of the feature value and user attributes.

### SUMMARY

In Patent Publication JP-A-2016-131825, systolic blood pressure is calculated by performing linear regression analysis using pulse wave propagation time, heart beat intervals, basic attributes such as age, body type attributes such as a BMI value, and cardiovascular attributes such as total blood cholesterol values, as explanatory variables, and using systolic blood pressure as an objective variable.

Calculating blood pressure on the basis of pulse wave velocity is advantageous in that no cuff is used. This advantage may be exploited to continuously measure blood pressure for an ambulatory user. In such a case, the user is not always in a resting state and the relationship between blood pressure and pulse wave velocity and so forth becomes complex, and therefore the relationship is not easy to model.

In a case where the blood pressure is calculated by linear regression analysis, as in the information processing device disclosed in Patent Publication JP-A-2016-131825, some model must be assumed for the relationship between systolic blood pressure and pulse wave propagation times and so forth. In ambulatory users, however, situations may arise in which the assumed model does not apply, which in turn negates the validity of the blood pressure that is calculated.

Therefore, the present invention provides a blood pressure measuring device, a blood pressure measurement method and a blood pressure measurement program that allow calculating blood pressure properly even for ambulatory users.

A blood pressure measuring device according to an aspect of the present invention has: an electrocardiogram acquisition unit that acquires an electrocardiogram of a user; a pulse wave acquisition unit that acquires a pulse wave of the user; a first extraction unit that extracts a heart rate on the basis of the electrocardiogram; a second extraction unit that extracts a pulse wave velocity on the basis of the electrocardiogram and the pulse wave; a third extraction unit that extracts one or a plurality of feature values pertaining to the pulse wave, on the basis of the pulse wave; and a calculation unit that calculates blood pressure of the user from the heart rate, the pulse wave velocity and the one or plurality of feature values extracted for the user, by a learner that has learned, by nonparametric regression analysis, a relationship between blood pressure of each of a plurality of subjects and a heart rate, a pulse wave velocity and one or a plurality of feature values pertaining to a pulse wave extracted for each of the plurality of subjects.

In such an implementation there is provided a learner that has learned, by nonparametric regression analysis, a relationship between blood pressure and a heart rate, a pulse wave velocity and one or a plurality of feature values pertaining to a pulse wave; as a result, the blood pressure of the user can be calculated without assuming a physical model. In consequence, blood pressure can be calculated properly even in cases where the user is not in a resting state and the relationship between blood pressure and pulse wave velocity and so forth is difficult to model.

In the above implementation, the learner may have a first learner that has learned a relationship between systolic blood pressure of each of the plurality of subjects and a heart rate, a pulse wave velocity and one or a plurality of feature values pertaining to a pulse wave extracted for each of the plurality of subjects; and the calculation unit may calculate, by way of the first learner, systolic blood pressure of the user from the heart rate, the pulse wave velocity and the one or plurality of feature values extracted for the user.

Such an implementation allows calculating systolic blood pressure properly even in cases where the user is not in a resting state and a relationship between systolic blood pressure and pulse wave velocity and so forth is difficult to model.

In the above implementation, the learner may have a second learner that has learned a relationship between diastolic blood pressure of each of the plurality of subjects and a heart rate, a pulse wave velocity and one or a plurality of feature values pertaining to a pulse wave extracted for each of the plurality of subjects; and the calculation unit may calculate, by way of the second learner, diastolic blood pressure of the user from the heart rate, the pulse wave velocity and the one or plurality of feature values extracted for the user.

Such an implementation allows calculating diastolic blood pressure properly even in cases where the user is not in a resting state and a relationship between diastolic blood pressure and pulse wave velocity and so forth is difficult to model.

In the above implementation, the first extraction unit may detect R-wave peak points of the electrocardiogram, perform an outlier exclusion process on the R-wave peak points, and thereafter extract the heart rate on the basis of a time interval of R-wave peak points.

In such an implementation the heart rate is extracted on the basis of a time interval of R-wave peak points having had outliers excluded therefrom; fluctuation in heart rate extraction precision, as affected by the behavioral state of the user, is prevented as a result.

In the above implementation, the second extraction unit may detect a rising point of the pulse wave, a point of maximal slope of the pulse wave and a systolic peak point of the pulse wave, may perform an outlier exclusion process on the rising point of the pulse wave, the point of maximal pulse wave slope and the systolic peak point of the pulse wave, and thereafter may extract pulse wave velocity on the basis of a time interval of R-wave peak points and any one of the rising point of the pulse wave, the point of maximal pulse wave slope and the systolic peak point of the pulse wave.

In such an implementation, fluctuation in the extraction precision of pulse wave velocity, as affected by the behavioral state of the user, are thus prevented through extraction of the pulse wave velocity on the basis of a time interval of R-wave peak points and any one of the rising point of the pulse wave, the point of maximal pulse wave slope and the systolic peak point of the pulse wave, having had outliers excluded therefrom.

In the above implementation, the third extraction unit may detect a diastolic peak point of the pulse wave, may perform an outlier exclusion process on the diastolic peak point of the pulse wave, and thereafter may extract a crest ratio of the systolic peak point and the diastolic peak point of the pulse wave, a time interval of the systolic peak point and the diastolic peak point of the pulse wave, and a time interval of the rising point and the systolic peak point of the pulse wave.

In such an implementation, fluctuation in the extraction precision of feature values, as affected by the behavioral state of the user, is prevented through extraction of feature values of the pulse wave on the basis of the systolic peak point and the diastolic peak point of the pulse wave, and the rising point of the pulse wave, having had outliers excluded therefrom.

In the above implementation, the third extraction unit may detect a plurality of peak points of an acceleration pulse wave being the acceleration of the pulse wave, may perform an outlier exclusion process on the plurality of peak points of the acceleration pulse wave being the acceleration of the pulse wave, and thereafter may extract crest ratios and time intervals between the plurality of peak points of the acceleration pulse wave.

In such an implementation, fluctuation in the extraction precision of feature values, as affected by the behavioral state of the user, is prevented through extraction of feature values of the pulse wave on the basis of the plurality of peak points of the acceleration pulse wave, having had outliers excluded therefrom.

In the above implementation, the third extraction unit may compare a velocity pulse wave at rest, being the velocity of a pulse wave acquired when the user is at rest, and a velocity pulse wave during exercise, being the velocity of a pulse wave acquired when the user is exercising, and may perform an outlier exclusion process on the pulse wave acquired when the user is exercising, and thereafter may extract one or a plurality of feature values pertaining to the pulse wave acquired when the user is exercising.

In such an implementation, fluctuation in the feature value extraction precision, as affected by the motion state of the user, is prevented by extracting feature values pertaining to a pulse wave acquired during exercise, after execution of an outlier exclusion process for the pulse wave acquired during exercise.

In the above implementation, the learner may learn a relationship between the blood pressure of each of the plurality of subjects and a heart rate, a pulse wave velocity and one or a plurality of feature values pertaining to a pulse wave extracted for each of the plurality of subjects, by using any one of: K-nearest neighbor regression of using blood pressure of K (K is a natural number equal to or greater than 1) subjects selected in ascending order of distance between the heart rate, the pulse wave velocity and the one or plurality of feature values extracted for the user, and a heart rate, a pulse wave velocity and one or a plurality of feature values pertaining to a pulse wave extracted for each of the plurality of subjects; random forest regression of constructing a plurality of decision trees for deciding blood pressure on the basis of quantities randomly selected from among a heart rate, a pulse wave velocity and one or a plurality of feature values pertaining to a pulse wave extracted for each of the plurality of subjects, and using blood pressure obtained in accordance with the plurality of decision trees on the basis of the heart rate, the pulse wave velocity and the one or plurality of feature values extracted for the user; and support vector regression of using values of a kernel function obtained on the basis of a support vector belonging to a regression hyperplane worked out so that respective margins for a heart rate, a pulse wave velocity and one or a plurality of feature values pertaining to a pulse wave extracted for each of the plurality of subjects are maximized, and on the basis of the heart rate, the pulse wave velocity and the one or plurality of feature values extracted for the user.

In such an implementation, the learner performs nonparametric regression analysis of working out a relationship between an explanatory variable including a heart rate, a pulse wave velocity and one or a plurality of feature values pertaining to a pulse wave, and an objective variable including blood pressure, without assuming the function form that expresses the relationship between the explanatory variable and the objective variable; blood pressure can thus be calculated properly also in cases where the relationship between blood pressure and pulse wave velocity and so forth is difficult to model.

A method of measuring blood pressure according to an aspect of the present invention includes: a step of acquiring an electrocardiogram of a user; a step of acquiring a pulse wave of the user; a step of extracting a heart rate on the basis of the electrocardiogram; a step of extracting a pulse wave velocity on the basis of the electrocardiogram and the pulse wave; a step of extracting one or a plurality of feature values pertaining to the pulse wave, on the basis of the pulse wave; and a step of calculating blood pressure of the user from the heart rate, the pulse wave velocity and the one or plurality of feature values extracted for the user, by a learner that has learned, by nonparametric regression analysis, a relationship between blood pressure of each of a plurality of subjects and a heart rate, a pulse wave velocity and one or a plurality of feature values pertaining to a pulse wave extracted for each of the plurality of subjects.

In such an implementation there is provided a learner that has learned, by nonparametric regression analysis, a relationship between blood pressure and a heart rate, a pulse wave velocity and one or a plurality of feature values pertaining to a pulse wave; as a result, the blood pressure of the user can be calculated without assuming a physical model. In consequence, blood pressure can be calculated properly even in cases where the user is not in a resting state and the relationship between blood pressure and pulse wave velocity and so forth is difficult to model.

A blood pressure measurement program according to an aspect of the present invention causes a computer to function as: an electrocardiogram acquisition unit that acquires an electrocardiogram of a user; a pulse wave acquisition unit that acquires a pulse wave of the user; a first extraction unit that extracts a heart rate on the basis of the electrocardiogram; a second extraction unit that extracts a pulse wave velocity on the basis of the electrocardiogram and the pulse wave; a third extraction unit that extracts one or a plurality of feature values pertaining to the pulse wave, on the basis of the pulse wave; and a calculation unit that calculates blood pressure of the user from the heart rate, the pulse wave velocity and the one or plurality of feature values extracted for the user, by a learner that has learned, by nonparametric regression analysis, a relationship between blood pressure of each of a plurality of subjects and a heart rate, a pulse wave velocity and one or a plurality of feature values pertaining to a pulse wave extracted for each of the plurality of subjects.

In such an implementation there is provided a learner that has learned, by nonparametric regression analysis, a relationship between blood pressure and a heart rate, a pulse wave velocity and one or a plurality of feature values pertaining to a pulse wave; as a result, the blood pressure of the user can be calculated without assuming a physical model. In consequence, blood pressure can be calculated properly even in cases where the user is not in a resting state and the relationship between blood pressure and pulse wave velocity and so forth is difficult to model.

The present invention succeeds thus in providing a blood pressure measuring device, a blood pressure measurement method and a blood pressure measurement program that allow calculating blood pressure properly even for ambulatory users.

### BRIEF DESCRIPTION OF DRAWINGS

FIG. 1 is a diagram illustrating an overview of the configuration of a blood pressure measuring device according to an embodiment of the present invention;

FIG. 2 is a diagram illustrating the physical configuration of an information processing terminal according to an embodiment of the present invention;

FIG. 3 is a function block diagram of a blood pressure measuring device according to an embodiment of the present invention;

FIG. 4 is a flowchart illustrating a blood pressure calculation process by a blood pressure measuring device according to an embodiment of the present invention;

FIG. 5 is a flowchart illustrating a feature point extraction process by a blood pressure measuring device according to an embodiment of the present invention;

FIG. 6 is a flowchart illustrating a peak point determination process by a blood pressure measuring device according to an embodiment of the present invention;

FIG. 7 is a flowchart illustrating a peak point detection process by a blood pressure measuring device according to an embodiment of the present invention;

FIG. 8 is a flowchart illustrating a peak point outlier exclusion process by a blood pressure measuring device according to an embodiment of the present invention;

FIG. 9 is a flowchart illustrating a feature point detection process by a blood pressure measuring device according to an embodiment of the present invention;

FIG. 10 is a flowchart illustrating a feature point outlier exclusion process by a blood pressure measuring device according to an embodiment of the present invention;

FIG. 11 is a flowchart illustrating a pulse wave outlier exclusion process by a blood pressure measuring device according to an embodiment of the present invention;

FIG. 12 is a diagram illustrating waveforms of an electrocardiogram and of a pulse wave acquired by a blood pressure measuring device according to an embodiment of the present invention;

FIG. 13 is a first diagram illustrating feature values, of a pulse wave, extracted by a blood pressure measuring device according to an embodiment of the present invention;

FIG. 14 is a second diagram illustrating feature values, of a pulse wave, extracted by a blood pressure measuring device according to an embodiment of the present invention;

FIG. 15 is a third diagram illustrating feature values, of a pulse wave, extracted by a blood pressure measuring device according to an embodiment of the present invention;

FIG. 16 is a diagram illustrating a correspondence relationship of extraction targets and feature values of a pulse wave, extracted by a blood pressure measuring device according to an embodiment of the present invention;

FIG. 17 is a flowchart illustrating a learning process for training a learner of a blood pressure measuring device according to an embodiment of the present invention; and

FIG. 18 is a flowchart illustrating a blood pressure calculation process by a blood pressure measuring device according to an embodiment of the present invention.

### DETAILED DESCRIPTION

Embodiments of the present invention will be explained next with reference to accompanying drawings. In the drawings, identical reference symbols denote identical or similar features.

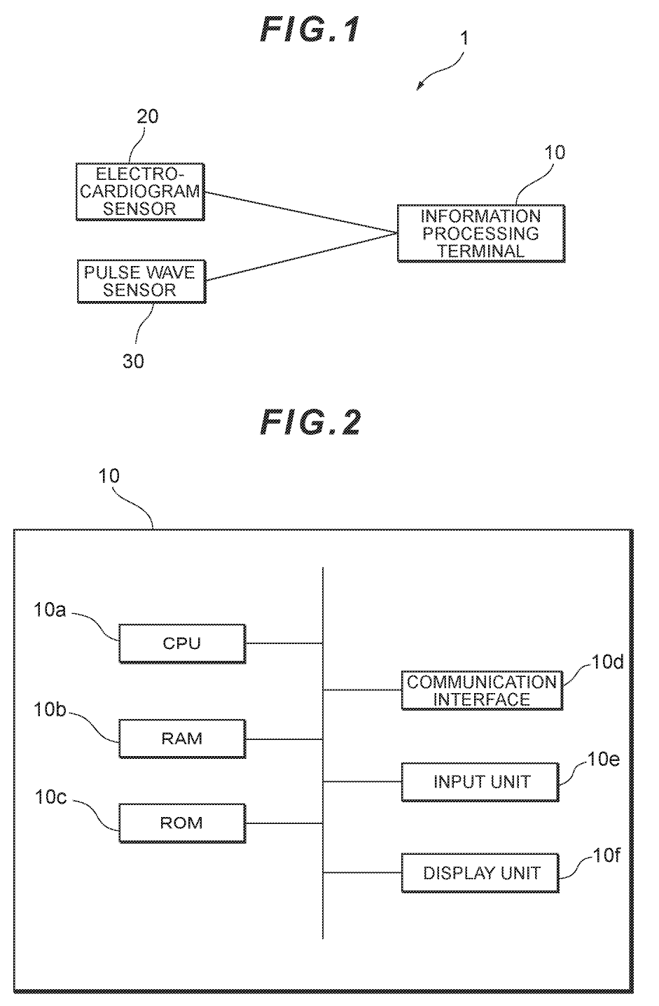

FIG. 1 illustrates an overview of the configuration of a blood pressure measuring device 1 according to an embodiment of the present invention. The blood pressure measuring device 1 has an information processing terminal 10, an electrocardiogram sensor 20 and a pulse wave sensor 30. The information processing terminal 10 acquires an electrocardiogram of a user measured by the electrocardiogram sensor 20 and a pulse wave of the user measured by the pulse wave sensor 30, performs a process of extracting various feature values from the electrocardiogram and from the pulse wave, and calculates the blood pressure of the user. The information processing terminal 10 may be a dedicated or general-purpose computer, and may be for instance a smartphone having a dedicated application installed thereon.

The electrocardiogram sensor 20 measures an electrocardiogram of the user by means of electrodes attached to the body of the user, and transmits the measured electrocardiogram to the information processing terminal 10 via wireless communication or wired communication. The pulse wave sensor 30 measures the arterial volume of the user by means of a light-emitting unit such as a light emitting diode (LED) and a light-receiving unit such as a photodiode (PD) or the like fitted to an ear or fingertip of the user, and transmits the measured arterial volume to the information processing terminal 10 by wireless communication or wired communication. The pulse wave sensor 30 according to the present embodiment is a transmissive photoelectric pulse wave sensor that receives light passing through sites of thin living tissue, such as the ear or the fingertips, and measures arterial volume on the basis of an absorption spectrum. However, a reflective photoelectric pulse wave sensor, other than a transmissive photoelectric pulse wave sensor, can be used herein as the pulse wave sensor 30, and other arbitrary pulse wave sensors can be used as well. The electrocardiogram sensor 20 and the pulse wave sensor 30 may be dedicated or general-purpose sensors. The blood pressure measuring device 1 may be a smart watch or wearable device in which the information processing terminal 10, the electrocardiogram sensor 20 and the pulse wave sensor 30 are partially or completely integrated with each other.

FIG. 2 is a diagram illustrating the physical configuration of the information processing terminal 10 according to an embodiment of the present invention. The information processing terminal 10 has a central processing unit (CPU) 10a corresponding to a hardware processor, a random access memory (RAM) 10b corresponding to a memory, a read only memory (ROM) 10c corresponding to a memory, a communication interface 10d, an input unit 10e, and a display unit 10f. These structures are connected by way of a bus so as to be capable of exchanging data with each other.

The CPU 10a executes programs stored in the RAM 10b or the ROM 10c, and computes and processes data. The CPU 10a is a computing device that executes an application for calculating the blood pressure of the user. The CPU 10a receives various input data from the input unit 10e and the communication interface 10d, displays the computation result of the input data on the display unit 10f, and stores the computation result in the RAM 10b and/or the ROM 10c.

The RAM 10b is a storage unit on which data can be rewritten, and is for instance made up of a semiconductor storage element. The RAM 10b stores data and programs such as applications that are executed by the CPU 10a.

The ROM 10c is a storage unit that allows only for data reading, and is for instance made up of a semiconductor storage element. The ROM 10c stores for instance programs and data such as firmware.

The communication interface 10d is a hardware interface that connects the information processing terminal 10 to the electrocardiogram sensor 20 and the pulse wave sensor 30, and may be a wireless communication interface or a wired communication interface. The communication interface 10d may connect the information processing terminal 10 to an external communication network such as the internet.

The input unit 10e receives input of data from the user, and is for instance made up of a keyboard and a mouse, or a touch panel.

The display unit 10f displays visually computation results by the CPU 10a, and is for instance made up of a liquid crystal display (LCD).

The information processing terminal 10 may be configured through execution of a blood pressure measurement program according to the present embodiment, by the CPU 10a of an ordinary smartphone or personal computer. The blood pressure measurement program may be stored and provided on a computer-readable storage medium, for instance the RAM 10b, ROM 10c or the like, or may be provided via an external communication network, such as the internet, connected by the communication interface 10d.

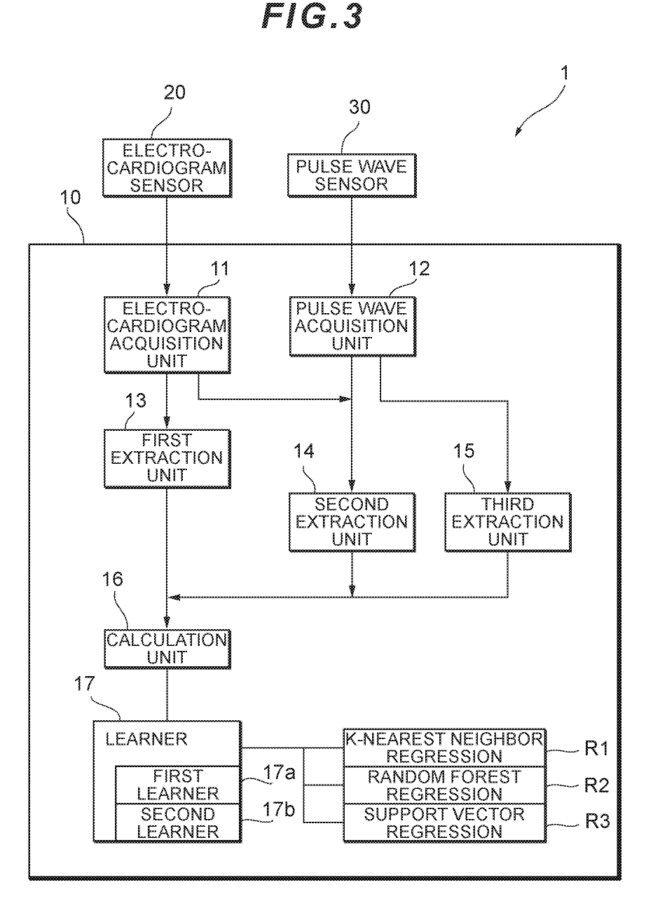

FIG. 3 is a function block diagram of the blood pressure measuring device 1 according to an embodiment of the present invention. The blood pressure measuring device 1 has an electrocardiogram acquisition unit 11, a pulse wave acquisition unit 12, a first extraction unit 13, a second extraction unit 14, a third extraction unit 15, a calculation unit 16 and a learner 17.

The electrocardiogram acquisition unit 11 acquires the electrocardiogram of the user as measured by the electrocardiogram sensor 20. The electrocardiogram acquisition unit 11 acquires continuously the electrocardiogram of the user and stores the electrocardiogram in a storage unit such as the RAM 10b. The pulse wave acquisition unit 12 acquires a pulse wave user as measured by the pulse wave sensor 30. The pulse wave acquisition unit 12 acquires continuously the pulse wave of the user, and stores the pulse wave in a storage unit such as the RAM 10b.

The first extraction unit 13 extracts the heart rate of the user on the basis of the acquired electrocardiogram of the user. As explained in detail further on, the first extraction unit 13 detects R-wave peak points of the electrocardiogram, performs an outlier exclusion process on the R-wave peak points, and thereafter extracts the heart rate on the basis of a time interval of R-wave peak points.

The second extraction unit 14 extracts a pulse wave velocity on the basis of the acquired electrocardiogram and pulse wave of the user. As explained in detail further on, the second extraction unit 14 detects a rising point of the pulse wave, a point of maximal pulse wave slope, and a systolic peak point of the pulse wave, performs an outlier exclusion process on the rising point of the pulse wave, the point of maximal pulse wave slope and the systolic peak point of the pulse wave, and thereafter extracts pulse wave velocity on the basis of the time interval of the R-wave peak points of the electrocardiogram and any one of the rising point of the pulse wave, the point of maximal pulse wave slope and the systolic peak point of the pulse wave.

The third extraction unit 15 extracts one or a plurality of feature values pertaining to a pulse wave on the basis of the acquired pulse wave of the user. As explained in detail further on, the third extraction unit 15 detects a diastolic peak point of the pulse wave, performs an outlier exclusion process on the diastolic peak point of the pulse wave, and thereafter extracts a crest ratio of the systolic peak point and the diastolic peak point of the pulse wave, a time interval of the systolic peak point and the diastolic peak point of the pulse wave, and a time interval of the rising point and the systolic peak point of the pulse wave. The third extraction unit 15 detects a plurality of peak points of an acceleration pulse wave being the acceleration of the pulse wave, performs an outlier exclusion process on the plurality of peak points of the acceleration pulse wave being the acceleration of the pulse wave, and thereafter extracts crest ratios and time intervals between the plurality of peak points of the acceleration pulse wave. Further, the third extraction unit 15 compares a velocity pulse wave at rest, being the velocity of a pulse wave acquired when the user is at rest, and a velocity pulse wave during exercise, being the velocity of the pulse wave acquired when the user is exercising, performs an outlier exclusion process on the pulse wave acquired when the user is exercising, and thereafter extracts one or a plurality of feature values pertaining to the pulse wave acquired when the user is exercising.

The calculation unit 16 calculates the blood pressure of the user from the heart rate, pulse wave velocity and one or a plurality of feature values extracted for the user, by the learner 17 that has learned, by nonparametric regression analysis, a relationship between blood pressure of each of a plurality of subjects and a heart rate, a pulse wave velocity and one or a plurality of feature values pertaining to a pulse wave extracted for each of the plurality of subjects. Herein nonparametric regression analysis refers to regression analysis in which a relationship between an explanatory variable and an objective variable is worked out without assuming the function form that expresses the relationship between the explanatory variable and the objective variable. Specifically, nonparametric regression analysis is herein regression analysis in which, when a heart rate, a pulse wave velocity and one or a plurality of feature values pertaining to a pulse wave are set as explanatory variables, and when blood pressure is set as the objective variable, a relationship between the blood pressure and the heart rate, the pulse wave velocity and the one or plurality of feature values pertaining to the pulse wave is worked out without assuming a physical model that expresses the relationship between the two variables. By contrast, in parametric regression analysis there is assumed the function form that expresses the relationship between the explanatory variable and the objective variable, and parameters included in the function are adjusted. Hyperparameters must be established also in nonparametric regression analysis.

The learner 17 has a first learner 17a that has learned a relationship between systolic blood pressure of each of a plurality of subjects and a heart rate, a pulse wave velocity and one or a plurality of feature values pertaining to a pulse wave extracted for each of the plurality of subjects, and a second learner 17b that has learned a relationship between diastolic blood pressure of the plurality of subjects and a heart rate, a pulse wave velocity and one or a plurality of feature values pertaining to a pulse wave extracted for each of the plurality of subjects. The calculation unit 16 calculates, by way of the first learner 17a, the systolic blood pressure of the user, from the heart rate, pulse wave velocity and one or a plurality of feature values extracted for the user, and calculates, by way of the second learner 17b, the diastolic blood pressure of the user from heart rate, pulse wave velocity and one or a plurality of feature values extracted for the user.

The learner 17 has learned a relationship between blood pressure of each of a plurality of subjects and a heart rate, a pulse wave velocity and one or a plurality of feature values pertaining to a pulse wave extracted for each of the plurality of subjects, by using any one of K-nearest neighbor regression R1, random forest regression R2 and support vector regression R3. Herein, K-nearest neighbor regression R1 utilizes the blood pressure of K (K is a natural number equal to or greater than 1) subjects selected in ascending order of distance between the heart rate, pulse wave velocity and one or a plurality of feature values extracted for the user and a heart rate, a pulse wave velocity and one or a plurality of feature values pertaining to a pulse wave extracted for each of the plurality of subjects. The distance between the heart rate, the pulse wave velocity and the one or plurality of feature values extracted for the user, and a heart rate, a pulse wave velocity and one ora plurality of feature values pertaining to a pulse wave extracted for each of the plurality of subjects may be the Euclidean distance in the respective spaces of the heart rate, pulse wave velocity and one or plurality of feature values, or may be any other distance. In K-nearest neighbor regression R1, a weighted average corresponding to the distance between the blood pressure of K subjects selected in ascending order of distance between the heart rate, the pulse wave velocity and the one or plurality of feature values extracted for the user, and a heart rate, a pulse wave velocity and one or a plurality of feature values pertaining to a pulse wave extracted for each of the plurality of subjects, may be taken as the calculated value of the blood pressure of the user, but the blood pressure of the user may be calculated using any function that has, as an argument, the blood pressure of K selected people. In K-nearest neighbor regression R1, the value of K or the coefficients of a weighting function for calculating the weighted average are hyperparameters, and can be set to arbitrary values.

In random forest regression R2 there is constructed a plurality of decision trees for deciding blood pressure on the basis of quantities randomly selected from among a heart rate, a pulse wave velocity and one or a plurality of feature values pertaining to a pulse wave extracted for each of a plurality of subjects, and there is used blood pressure worked out according to the plurality of decision trees on the basis of the heart rate, pulse wave velocity and one or a plurality of feature values extracted for the user. In random forest regression R2 the average value of blood pressure worked out according to the plurality of decision trees may be used as the calculated value of the blood pressure of the user, but the blood pressure of the user may be calculated using any function that has, as an argument, blood pressure worked out according to the plurality of decision trees. In random forest regression R2 the number of constituent decision trees and the number of nodes (decision tree depth) included in each decision tree are hyperparameters, and can be set to arbitrary values.

In support vector regression R3 there are used values of a kernel function obtained on the basis of a support vector belonging to a regression hyperplane worked out so that respective margins for a heart rate, a pulse wave velocity and one or a plurality of feature values pertaining to a pulse wave having been extracted for each of a plurality of subjects are maximized, and on the basis of the heart rate, pulse wave velocity and one or a plurality of feature values extracted for the user. The margin is herein the smallest distance from among the vertical distances between the regression hyperplane and the points denote a heart rate, a pulse wave velocity and one or a plurality of feature values pertaining to a pulse wave. The distance may be measured according to an Euclidean distance in the respective spaces representing the heart rate, pulse wave velocity and one or plurality of feature values pertaining to the pulse wave. In support vector regression R3 a cost parameter for determining the regression hyperplane and the coefficients of the kernel function are hyperparameters, and can be set to arbitrary values.

Learning by the learner 17 is executed beforehand on the basis of the blood pressure and a heart rate, a pulse wave velocity and one or a plurality of feature values pertaining to a pulse wave extracted for each of the plurality of subjects, whereupon the trained learner 17 is installed in the information processing terminal 10. The information processing terminal 10 may receive updates of the learner 17 via an external communication network such as the internet.

The learner 17 utilizes any one of K-nearest neighbor regression R1, random forest regression R2 and support vector regression R3 to perform nonparametric regression analysis of working out a relationship between an explanatory variable including a heart rate, a pulse wave velocity and one or a plurality of feature values pertaining to a pulse wave, and an objective variable including blood pressure, without assuming a function form that expresses the relationship between the explanatory variable and the objective variable. As a result the blood pressure measuring device 1 can calculate blood pressure properly also in cases where the relationship between blood pressure and pulse wave velocity and so forth is difficult to model.

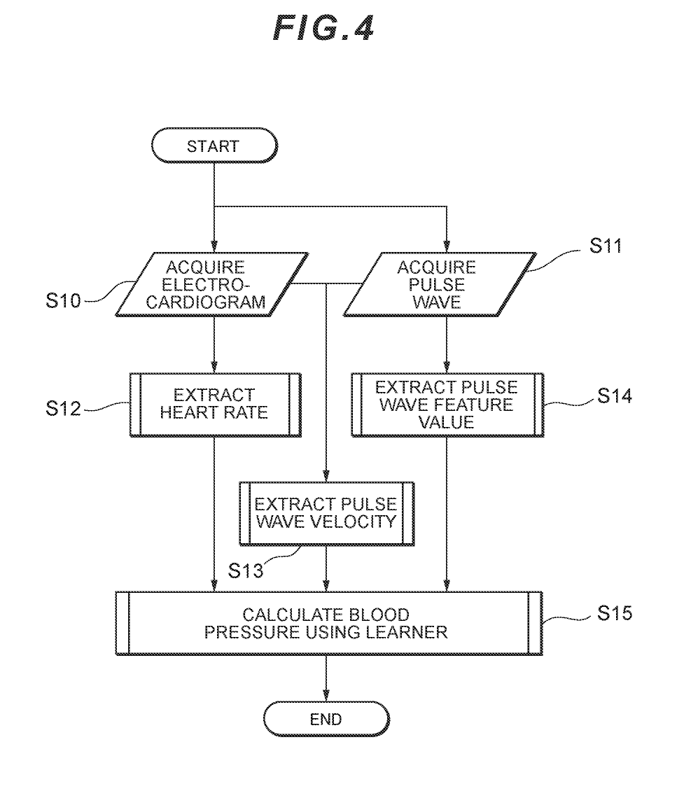

FIG. 4 is a flowchart illustrating a blood pressure calculation process by the blood pressure measuring device 1 according to an embodiment of the present invention. The blood pressure measuring device 1 acquires an electrocardiogram of the user by way of the electrocardiogram sensor 20 (S10), and acquires a pulse wave of the user by way of the pulse wave sensor 30 (S11). The electrocardiogram and the pulse wave are acquired continuously.

Next, the blood pressure measuring device 1 extracts a heart rate on the basis of the electrocardiogram (S12). The blood pressure measuring device 1 extracts a pulse wave velocity on the basis of the electrocardiogram and the pulse wave (S13). Further, the blood pressure measuring device 1 extracts one or a plurality of feature values pertaining to the pulse wave, on the basis of the pulse wave (S14).

Lastly, the blood pressure measuring device 1 calculates the blood pressure of the user from the heart rate, pulse wave velocity and one or a plurality of feature values extracted for the user, by the learner 17 that has learned, by nonparametric regression analysis, a relationship between blood pressure of each of a plurality of subjects and a heart rate, a pulse wave velocity and one or a plurality of feature values pertaining to a pulse wave extracted for each of the plurality of subjects (S15).

The blood pressure measuring device 1 according to the present embodiment is provided with the learner 17 that has learned, by nonparametric regression analysis, a relationship between blood pressure and a heart rate, a pulse wave velocity and one or a plurality of feature values pertaining to a pulse wave; as a result, the blood pressure measuring device 1 can calculate the blood pressure of the user without assuming a physical model. In consequence, blood pressure can be calculated properly even in cases where the user is not in a resting state and the relationship between blood pressure and pulse wave velocity and so forth is difficult to model.

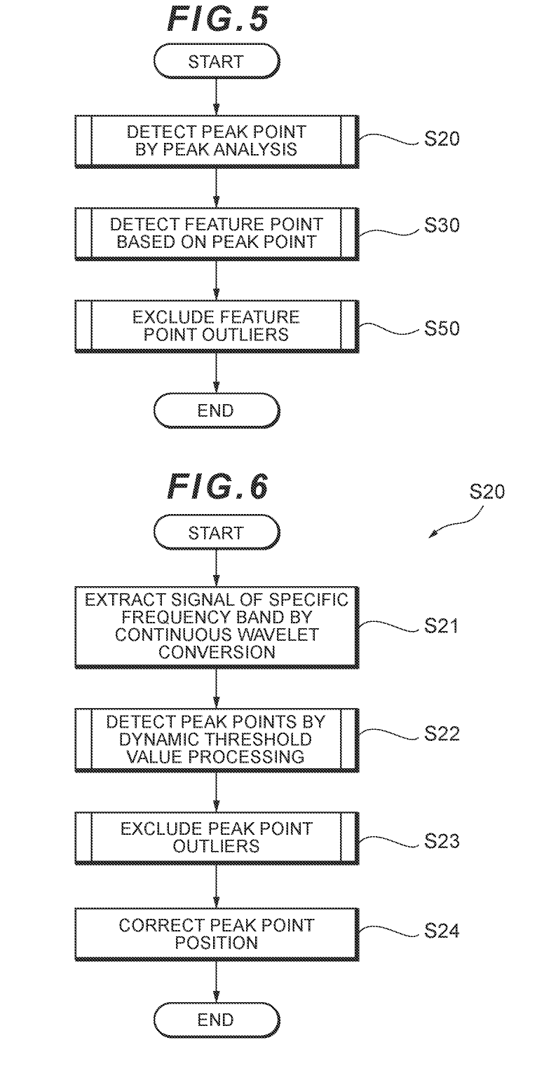

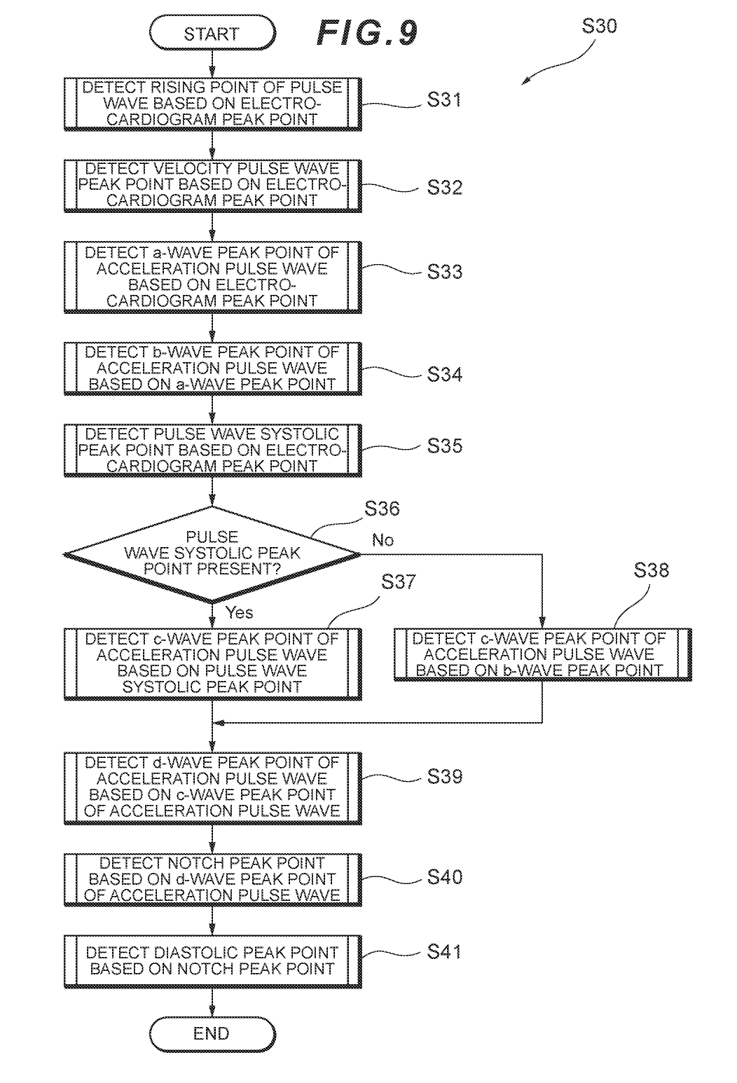

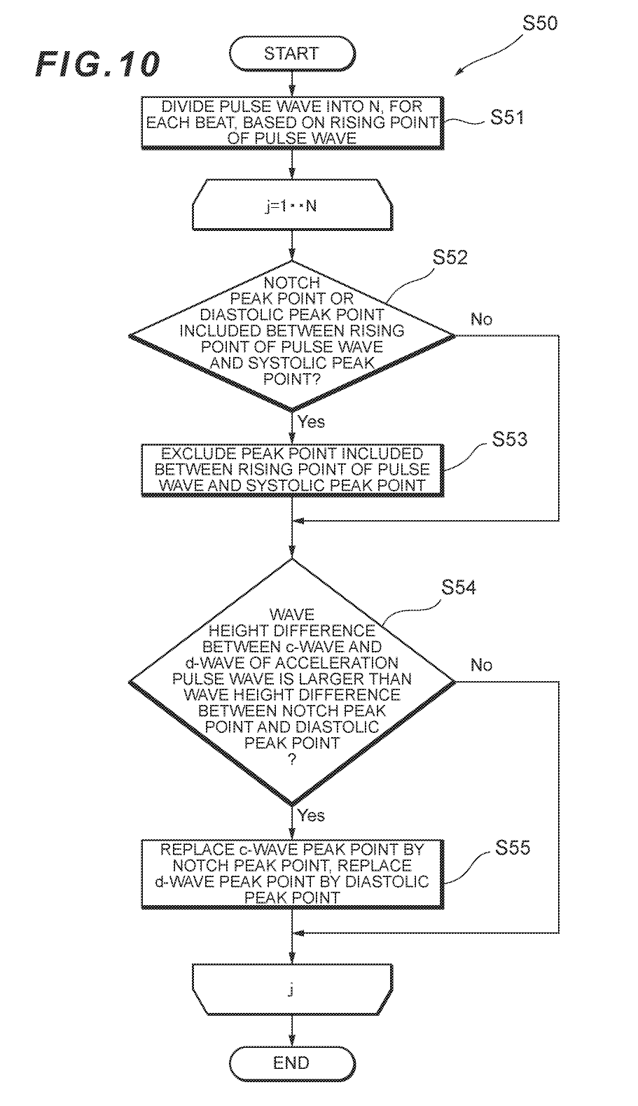

FIG. 5 is a flowchart illustrating a feature point extraction process by the blood pressure measuring device 1 according to an embodiment of the present invention. The feature point extraction process is a process executed for the acquired electrocardiogram and for the acquired pulse wave. In the feature point extraction process, firstly an acquired waveform is subjected to peak analysis, to detect peak points (S20). Peak point detection (S20) will be explained in detail with reference to FIG. 6 to FIG. 8. Next, feature points are extracted based on the detected peak points (S30). Feature point extraction (S30) will be explained in detail with reference to FIG. 9. Lastly, there is executed a feature point outlier exclusion process (S50). Outlier exclusion (S50) will be explained in detail with reference to FIG. 10.

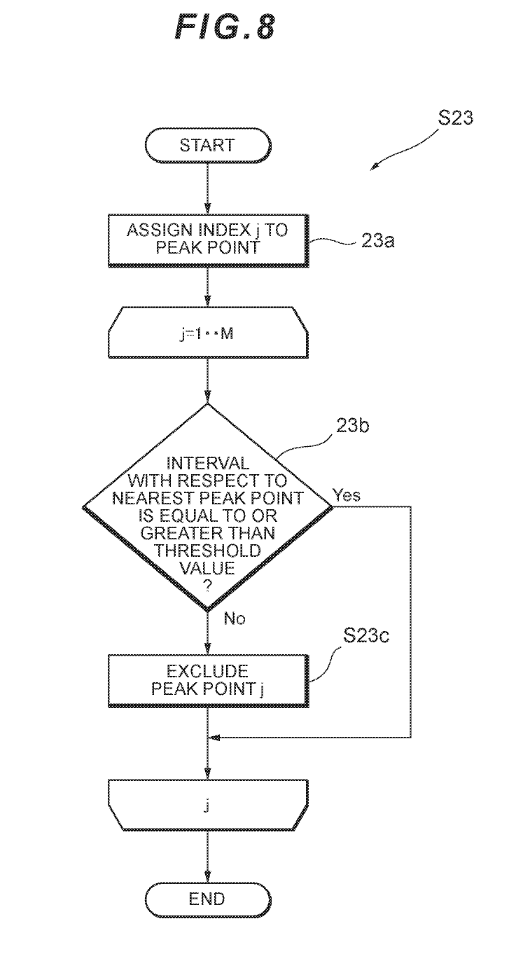

FIG. 6 is a flowchart illustrating a peak point determination process by the blood pressure measuring device 1 according to an embodiment of the present invention. FIG. 6 is a flowchart illustrating the details of the peak point detection process (S20) depicted in FIG. 5. In the peak point detection process (S20), firstly the waveform is subjected to continuous wavelet conversion, to extract a signal of a specific frequency band (S21). Herein continuous wavelet conversion is carried out by adjusting as appropriate a scaling factor and a time shift factor, using for instance a Mexican hat-type function as a mother wavelet function. Waveform peak points are detected next by resorting to a dynamic threshold value process (S22). This process will be explained in detail with reference to figures below. Peak point outliers are excluded thereafter (S23). This process will be explained in detail with reference to FIG. 8. Peak positions are corrected last (S24), whereby the peak point determination process is over.

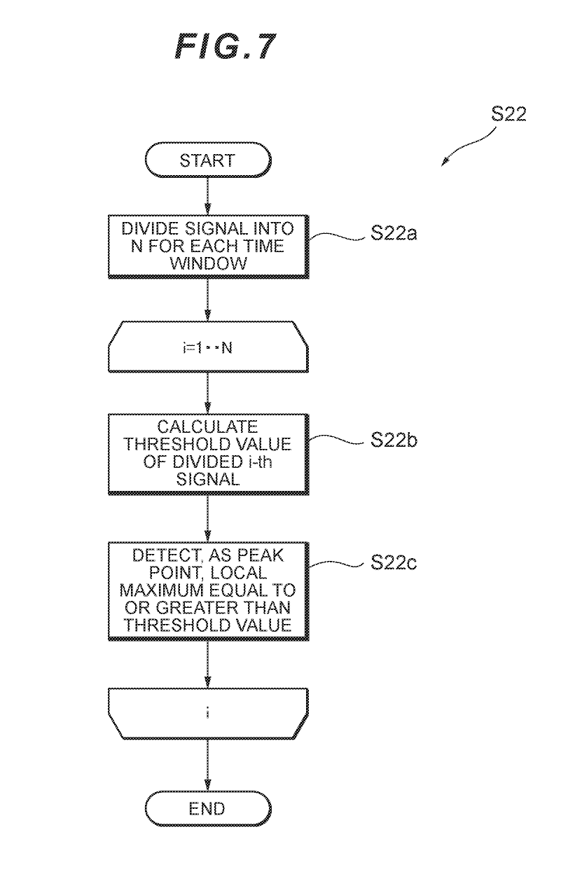

FIG. 7 is a flowchart illustrating a peak point detection process by the blood pressure measuring device 1 according to an embodiment of the present invention. FIG. 7 is a flowchart illustrating the details of the peak point detection process (S22) in which the dynamic threshold value process depicted in FIG. 6 is resorted to. In the peak point detection process (S22) that utilizes dynamic threshold value process, firstly the signal resulting from continuous wavelet conversion is divided into N, for each time window (S22a). Herein N is a natural number that can be set arbitrarily. Subsequently, there is calculated a threshold value of an i-th (I=1 to N) signal from among the N divided signals (S22b). The threshold value may be set to s % of the signal amplitude or to t % of the maximum value of the signal, in the time window. Herein s and t are real numbers lying in the range of 0 to 100, and are adjusted so as to enable optimal peak detection. To detect the rising point of the pulse wave, the threshold value may be set to t % of the minimum value of the signal in the time window. After calculation of the threshold value, a local maximum equal to or greater than the threshold value is detected as a peak point (S22c). A minimum point equal to or smaller than the threshold value is detected, as a peak point, to detect the rising point of the pulse wave. The peak point detection process (S22) utilizing a dynamic threshold value process, as described above, is thereby over.

FIG. 8 is a flowchart illustrating a peak point outlier exclusion process by the blood pressure measuring device 1 according to an embodiment of the present invention. FIG. 8 is a flowchart illustrating the details of the peak point outlier exclusion process (S23) depicted in FIG. 6. In the peak point outlier exclusion process (S23) firstly an index j is assigned to respective peak points (S23a). The index j may be assigned in descending order of peak point amplitude. Herein it will be assumed that there are M peak points. It is then determined, for each peak point to which the index j is assigned, whether or not an interval with respect to a nearest peak point is equal to or greater than a threshold value (S23b). If the interval with respect to the nearest peak point is not equal to or greater than the threshold value (S23b: No), i.e. if the interval between the j-th peak point and the nearest peak point is smaller than the threshold value, the j-th peak point is excluded (S23c). In the case for instance where the peak point is an R-wave peak point of an electrocardiogram, the threshold value may be set to 0.05 seconds and the process of excluding a j-th R-wave peak point may be performed if the R-wave peak point adjacent to the j-th R-wave peak point is shorter than a 0.05 second interval. The peak point outlier exclusion process (S23) is thereby over.

FIG. 9 is a flowchart illustrating a feature point detection process by the blood pressure measuring device 1 according to an embodiment of the present invention. FIG. 9 is a flowchart illustrating the details of the feature point detection process (S30) depicted in FIG. 5. The feature point detection process (S30) is a process of detecting a plurality of feature points of a pulse wave. Firstly, a rising point P10 of a pulse wave is detected on the basis of electrocardiogram peak points (S31). Electrocardiogram peak points are R-wave peak points in the electrocardiogram. Next, a peak point of a velocity pulse wave is detected based on the electrocardiogram peak points, for a velocity pulse wave being the first derivative of the pulse wave waveform with respect to time (S32). The peak point of the velocity pulse wave is the point of maximal pulse wave slope P11. Further, an a-wave peak point P20 of an acceleration pulse wave, being the second derivative of the pulse wave waveform with respect to time, is detected based on the electrocardiogram peak points (S33). As explained below, the acceleration pulse wave includes typically an a-wave, a b-wave, a c-wave, a d-wave and an e-wave.

Next a b-wave peak point P21 of the acceleration pulse wave is detected based on the a-wave peak point P20 (S34). A systolic peak point P12 of the pulse wave is detected based on the electrocardiogram peak points (S35). The systolic peak point P12 of the pulse wave may in some instances be absent. For this reason, it is determined whether a systolic peak point P12 of the pulse wave is present or not (S36). If a systolic peak point P12 is present (S36: Yes), a c-wave peak point P22 of the acceleration pulse wave is detected based on the systolic peak point P12 (S37). If on the other hand no systolic peak point P12 is present (S36: No), the c-wave peak point P22 is detected based on the b-wave peak point P21 (S38).

Further, a d-wave peak point P23 of the acceleration pulse wave is detected based on the c-wave peak point P22 of the acceleration pulse wave (S39), and a notch peak point P14 is detected based on the d-wave peak point P23 of the acceleration pulse wave (S40). Lastly, a diastolic peak point P13 is detected based on the notch peak point P14 (S41). The feature point detection process (S30) is thereby over.

FIG. 10 is a flowchart illustrating a feature point outlier exclusion process by the blood pressure measuring device 1 according to an embodiment of the present invention. FIG. 10 is a flowchart illustrating the details of the feature point outlier exclusion process (S50) depicted in FIG. 5. The feature point outlier exclusion process (S50) is a process of excluding outliers of detected feature points of the pulse wave. Firstly, the pulse wave is divided into N for each beat, based on the rising point P10 of the pulse wave (S51). An index j (j=1 to N) is assigned to each divided pulse wave. Next it is determined whether the j-th divided pulse wave includes or not the notch peak point P14 or the diastolic peak point P13 between the rising point P10 and the systolic peak point P12 (S52). If the notch peak point P14 or diastolic peak point P13 is included between the rising point P10 and the systolic peak point P12 (S52: Yes), the peak point included between the rising point P10 and the systolic peak point P12 is excluded (S53). As a result it becomes possible to prevent misdetection of peak points in a subsequent pulse wave, also in a case where the notch peak point P14 or the diastolic peak point P13 fails to be properly detected.

In a case where no notch peak point P14 or diastolic peak point P13 is included between the rising point P10 of the pulse wave and the systolic peak point P12 (S52: No), as well as after exclusion of a peak point included between the rising point P10 and the systolic peak point P12 (S53), it is determined whether a wave height difference of the c-wave and the d-wave of the acceleration pulse wave is larger or not than the wave height difference of the notch peak point P14 and the diastolic peak point P13 (S54). If it is determined that the wave height difference between the c-wave and the d-wave of the acceleration pulse wave is larger than the wave height difference of the notch peak point P14 and the diastolic peak point P13 (S54: Yes), the c-wave peak point is replaced by the notch peak point P14 and the d-wave peak point is replaced by the diastolic peak point P13 (S55). As a result there is prevented misdetection of the c-wave peak point or the d-wave peak point of the acceleration pulse wave, and feature values of the pulse wave are properly extracted. In a case where it is determined that the wave height difference of the c-wave and the d-wave of the acceleration pulse wave is equal to or smaller than the wave height difference of the notch peak point P14 and the diastolic peak point P13 (S54: No), as well as after replacement of the c-wave peak point by the notch peak point P14 and replacement of the d-wave peak point by the diastolic peak point P13 (S55), a similar process is executed for the (j+1)-th divided pulse wave. The feature point outlier exclusion process (S50) is thereby over.

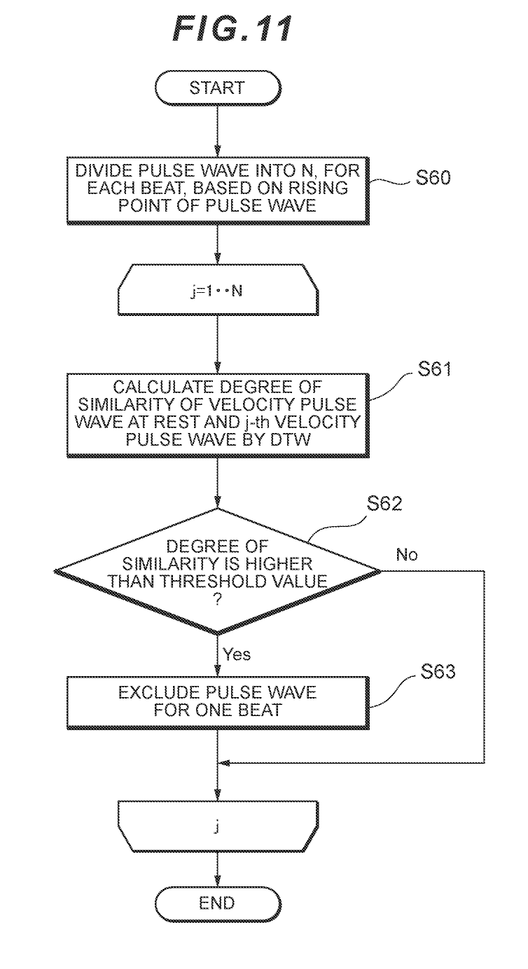

FIG. 11 is a flowchart illustrating a pulse wave outlier exclusion process by the blood pressure measuring device 1 according to an embodiment of the present invention. In some instances the pulse wave acquired during exercise includes pulse wave disturbances caused by body movements. The pulse wave outlier exclusion process is a process of excluding outliers included in a pulse wave thus disturbed.

In the pulse wave outlier exclusion process, firstly the pulse wave is divided into N for each beat, based on the rising point P10 of the pulse wave (S60). An indexj is assigned to each divided pulse wave. The same indexj is assigned to a corresponding velocity pulse wave. Next a degree of similarity of the velocity pulse wave at rest and the j-th velocity pulse wave is calculated by dynamic time warping (DTW) (S61). Herein DTW is a method for measuring the degree of similarity of two time-series data sets, wherein all combinations of distances between points included in two time-series data sets are compared, and the shortest path distance is taken as the degree of similarity. After calculation of the degree of similarity, it is determined whether the degree of similarity is higher than a threshold value or not (S62). If the degree of similarity is higher than the threshold value (S62: Yes), the pulse wave for one beat is excluded (S63). If on the other hand the degree of similarity is lower than the threshold value (S62: No), a similar process is executed for the (j+1)-th divided pulse wave. The pulse wave outlier exclusion process is thereby over. Fluctuation of the feature value extraction precision, as affected by the motion state of the user, can be thus prevented through extraction of feature values pertaining to a pulse wave acquired during exercise, after execution of the outlier exclusion process on for the pulse wave.

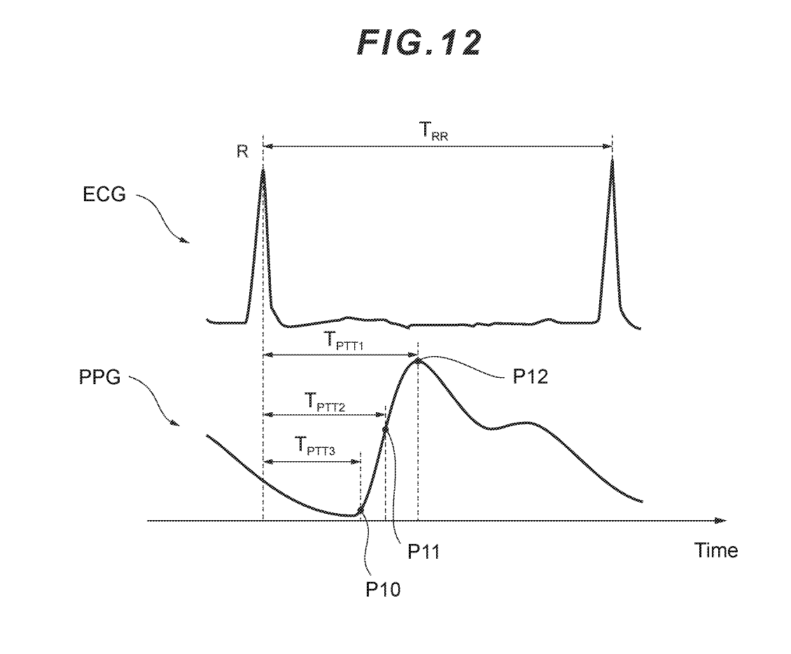

FIG. 12 is a diagram illustrating a waveform of an electrocardiogram ECG and of a pulse wave PPG acquired by the blood pressure measuring device 1 according to an embodiment of the present invention. FIG. 12 represents time in the horizontal axis and illustrates, above the axis, a waveform of an electrocardiogram ECG and, underneath, a waveform of a pulse wave PPG. The first extraction unit 13 detects R-wave peak points of the electrocardiogram ECG, performs an outlier exclusion process on the R-wave peak points, and thereafter extracts the heart rate on the basis of the time interval T_{RR} of the R-wave peak points. The heart rate is calculated as the reciprocal of the time interval T_{RR} of the R-wave peak points; the heart rate units may be bpm (beats per minute). The heart rate is extracted by the first extraction unit 13 on the basis of the time interval T_{RR} of R-wave peak points having had outliers excluded therefrom; fluctuation in heart rate extraction precision, as affected by the behavioral state of the user, is prevented as a result.

The second extraction unit 14 detects the rising point P10, the point of maximal pulse wave slope P11 and the systolic peak point P12 of the pulse wave PPG, performs an outlier exclusion process for the rising point P10, the point of maximal pulse wave slope P11 and the systolic peak point P12, and thereafter extracts pulse wave velocity on the basis of any one of a time interval T_{PTT3} of the rising point P10 and an R-wave peak point, a time interval T_{PTT2} of the point of maximal pulse wave slope P11 and the R-wave peak point, and a time interval T_{PTT1} of the systolic peak point P12 and the R-wave peak point. More specifically, the pulse wave velocity is calculated on the basis of any one of v₁=L/T_{PTT1}, v₂=L/T_{PTT2} and v₃=L/T_{PTT3}, where L denotes the height of the user.

Fluctuation in pulse wave velocity extraction precision, as affected by the behavioral state of the user, is thus prevented through extraction, by the second extraction unit 14, of the pulse wave velocity on the basis of a time interval of an R-wave peak point and any one of the rising point P10, the point of maximal pulse wave slope P11 and the systolic peak point P12 of the pulse wave PPG, having had outliers excluded therefrom.

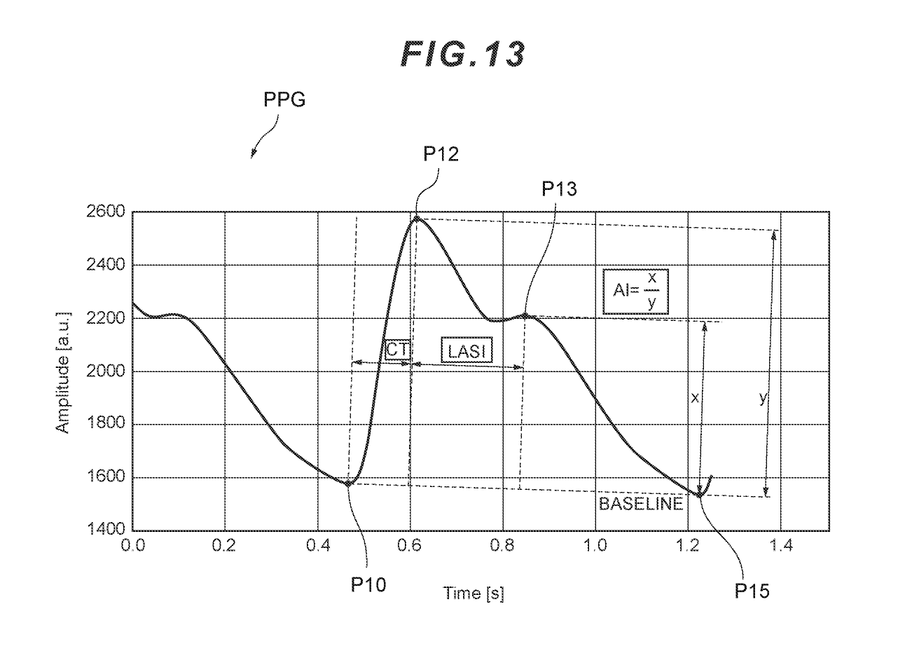

FIG. 13 is a first diagram illustrating feature values of the pulse wave PPG extracted by the blood pressure measuring device 1 according to an embodiment of the present invention. FIG. 13 represents time in the horizontal axis, and represents pulse wave amplitude in the vertical axis. The figure depicts crest time (CT), large artery stiffness index (LASI) and augmentation index (AI) as feature values of the pulse wave PPG. In the figure, the reference symbol “x” denotes the vertical distance from a baseline that joins the rising point P10 of the pulse wave PPG and a nearest rising point P15, up to the diastolic peak point P13. The reference symbol “y” denotes the vertical distance from the baseline up to the systolic peak point P12.

The third extraction unit 15 detects the diastolic peak point P13 of the pulse wave PPG, performs an outlier exclusion process on the diastolic peak point P13, and thereafter extracts the crest ratio of the systolic peak point P12 and the diastolic peak point P13, the time interval of the systolic peak point P12 and the diastolic peak point P13, and the time interval of the rising point P10 and the systolic peak point P12. The crest ratio of the systolic peak point P12 and the diastolic peak point P13, referred to as AI, is the ratio x/y of the vertical distance y from the baseline up to the systolic peak point P12 and the vertical distance x from the baseline up to the diastolic peak point P13. Herein AI may be used as an indicator that denotes organic vascular stiffening and changes in functional blood pressure. The time interval of the systolic peak point P12 and the diastolic peak point P13 is referred to as LASI. Herein LASI may be used as an indicator that denotes organic vascular stiffening and changes in functional blood pressure. The time interval of the rising point P10 and the systolic peak point P12 is referred to as CT. Herein CT may be used as an indicator for identifying a cardiovascular disease. Fluctuation in the feature value extraction precision, as affected by the behavioral state of the user, is prevented through extraction of feature values of the pulse wave, by the third extraction unit 15, on the basis of the systolic peak point P12, the diastolic peak point P13 and the rising point P10 of the pulse wave PPG, having had outliers excluded therefrom.

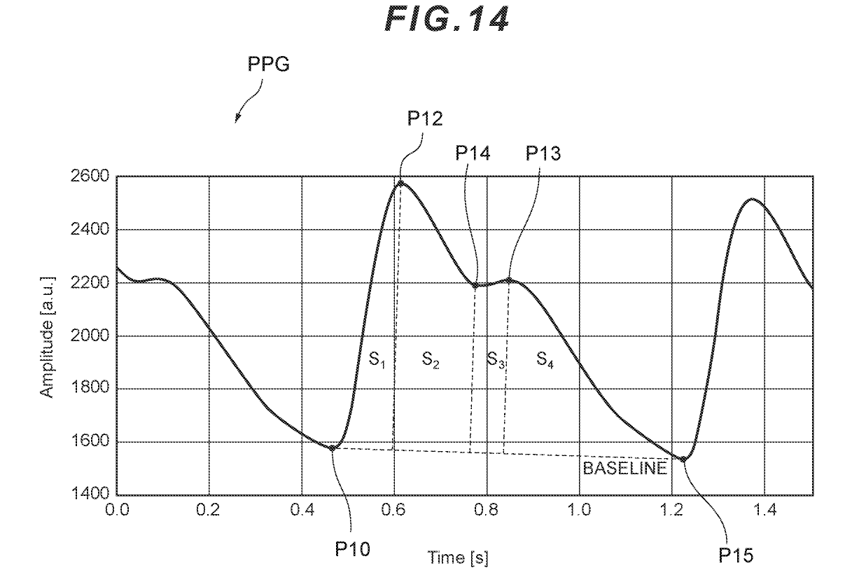

FIG. 14 is a second diagram illustrating feature values, of a pulse wave, extracted by the blood pressure measuring device 1 according to an embodiment of the present invention. FIG. 14 represents time in the horizontal axis and represents the amplitude of a pulse wave PPG in the vertical axis, and depicts, as quantities for calculating feature values of the pulse wave PPG: a surface area S1 enclosed by the baseline and the waveform from the rising point P10 up to the systolic peak point P12 of the pulse wave PPG, a surface area S2 enclosed by the baseline and the waveform from the systolic peak point P12 up to the notch peak point P14, a surface area S3 enclosed by the baseline and the waveform from the notch peak point P14 up to the diastolic peak point P13, and a surface area S4 enclosed by the baseline and the waveform from the diastolic peak point P13 up to the nearest rising point P15. The third extraction unit 15 extracts, as feature values of the pulse wave, a surface area ratio r_{S2S1}=S2/S1, a surface area ratio r_{S3S1}=S3/S1 and a surface area ratio r_{S4S1}=S4/S1. These surface area ratios are referred to as inflection point area ratios (IPAs), and may be used as indicators denoting an impedance characteristics of the peripheral vascular state.

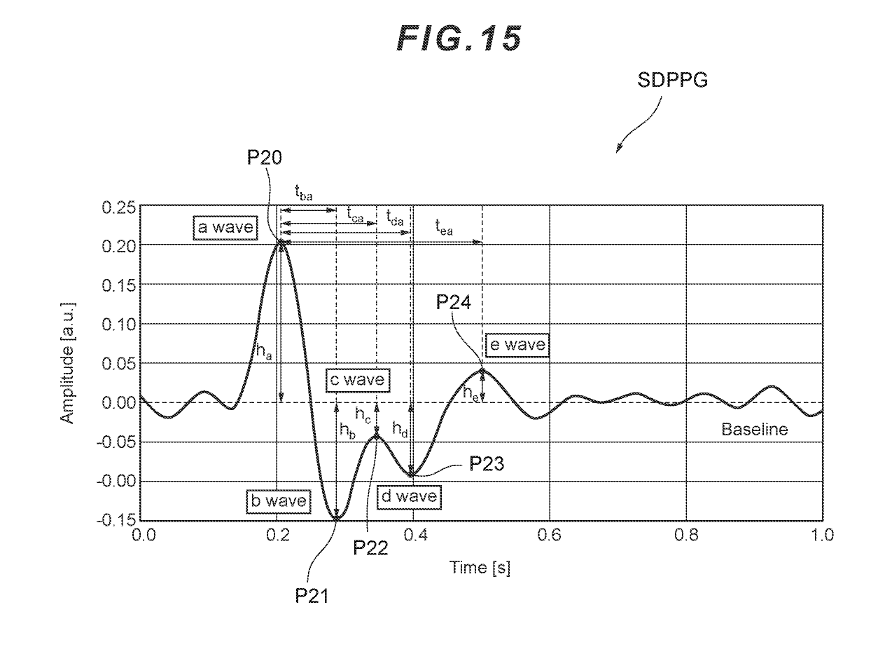

FIG. 15 is a third diagram illustrating feature values, of a pulse wave, extracted by the blood pressure measuring device 1 according to an embodiment of the present invention. FIG. 15 represents time in the horizontal axis and represents the amplitude of an acceleration pulse wave SDPPG in the vertical axis. FIG. 15 illustrates, as peak points for extracting feature values of the pulse wave PPG, an a-wave peak point P20, a b-wave peak point P21, a c-wave peak point P22, a d-wave peak point P23 and an e-wave peak point P24 of the acceleration pulse wave SDPPG. In FIG. 15, “hₐ” denotes the vertical distance from the baseline up to the a-wave peak point P20, i.e. the height of the a-wave. Similarly, “h_{b}” denotes the vertical distance from the baseline up to the b-wave peak point P21, “h_{c}” denotes the vertical distance from the baseline up to the c-wave peak point P22, “h_{d}” denotes the vertical distance from the baseline up to the d-wave peak point P23, and “hₑ” denotes the vertical distance from the baseline up to the e-wave peak point P24; further, “t_{ba}” denotes the time interval of the a-wave peak point P20 and the b-wave peak point P21, “t_{ca}” denotes the time interval of the a-wave peak point P20 and the c-wave peak point P22, “t_{da}” denotes the time interval of the a-wave peak point P20 and the d-wave peak point P23, and “tₑₐ” denotes the time interval of the a-wave peak point P20 and the e-wave peak point P24.

The third extraction unit 15 detects a plurality of peak points of an acceleration pulse wave SDPPG being the acceleration of the pulse wave PPG, executes an outlier exclusion process on the plurality of peak points of the acceleration pulse wave SDPPG, and thereafter extracts crest ratios and time intervals between the plurality of peak points of the acceleration pulse wave SDPPG. More specifically, the third extraction unit 15 extracts a crest ratio r_{ba}=h_{b}/hₐ of the a-wave and the b-wave, a crest ratio r_{ca}=h_{c}/hₐ of the a-wave and the c-wave, a crest ratio r_{da}=h_{d}/hₐ of the a-wave and the d-wave and a crest ratio rₑₐ=hₑ/hₐ of the a-wave and the e-wave. The third extraction unit 15 extracts a time interval t_{ba} of the a-wave and the b-wave, a time interval tca of the a-wave and the c-wave, a time interval t_{da} of the a-wave and the d-wave, and a time interval tₑₐ of the a-wave and the e-wave. These quantities may be used as indicators for estimating a vascular state. For instance, the crest ratio r_{ba} of the a-wave and the b-wave may be used as an indicator denoting organic vascular stiffening, and the crest ratio r_{da} of the a-wave and the d-wave may be used as an indicator denoting changes in functional blood pressure. Fluctuation in the feature value extraction precision, as affected by the behavioral state of the user, is thus prevented through extraction of feature values of the pulse wave PPG, by the third extraction unit 15, on the basis of the plurality of peak points of the acceleration pulse wave SDPPG having had outliers excluded therefrom.

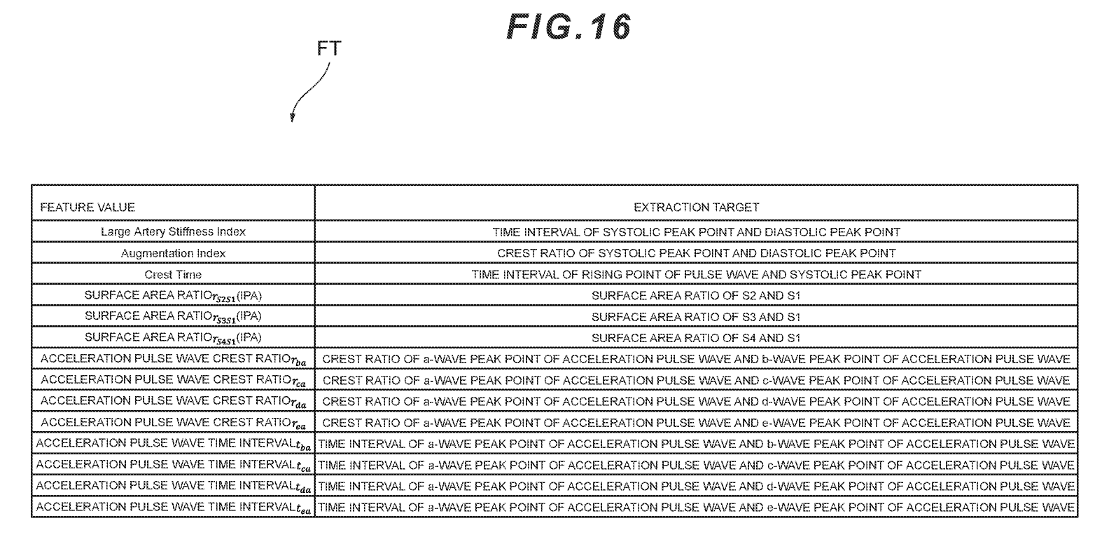

FIG. 16 is a diagram illustrating a correspondence relationship of extraction targets and feature values FT of the pulse wave PPG, extracted by the blood pressure measuring device 1 according to an embodiment of the present invention. In order from the top, the extraction target of LASI is the time interval of the systolic peak point P12 and the diastolic peak point P13, the extraction target of AI is the crest ratio of the systolic peak point P12 and the diastolic peak point P13, and the extraction target of CT is the time interval of the rising point P10 and the systolic peak point P12. Further, the extraction target of the surface area ratio r_{S2S1} is the surface area ratio of S2 and S1, the extraction target of the surface area ratio r_{S3S1} is the surface area ratio of S3 and S1, and the extraction target of the surface area ratio r_{S4S1} is the surface area ratio of S4 and S1. Further, the extraction target of the crest ratio r_{ba} of the a-wave and the b-wave is the crest ratio of the vertical distance hₐ from the baseline up to the a-wave peak point P20 and the vertical distance h_{b} from the baseline up to the b-wave peak point P21, the extraction target of the crest ratio r_{ca} of the a-wave and the c-wave is the crest ratio of the vertical distance hₐ from the baseline up to the a-wave peak point P20 and the vertical distance h_{c} from the baseline up to the c-wave peak point P22; the extraction target of the crest ratio r_{da} of the a-wave and the d-wave is the crest ratio of the vertical distance hₐ from the baseline up to the a-wave peak point P20 and the vertical distance h_{d} from the baseline up to the d-wave peak point P23; and the extraction target of the crest ratio rₑₐ of the a-wave and the e-wave is the crest ratio of the vertical distance hₐ from the baseline up to the a-wave peak point P20 and the vertical distance hₑ from the baseline up to the e-wave peak point P24. Further, the extraction target of the time interval t_{ba} of the a-wave and the b-wave is the time interval of the a-wave peak point P20 and the b-wave peak point P21, the extraction target of the time interval t_{ca} of the a-wave and the c-wave is the time interval of the a-wave peak point P20 and the c-wave peak point P22, the extraction target of the time interval t_{da} of the a-wave and the d-wave is the time interval of the a-wave peak point P20 and the d-wave peak point P23, and the extraction target of the time interval tₑₐ of the a-wave and the e-wave is the time interval of the a-wave peak point P20 and the e-wave peak point P24.

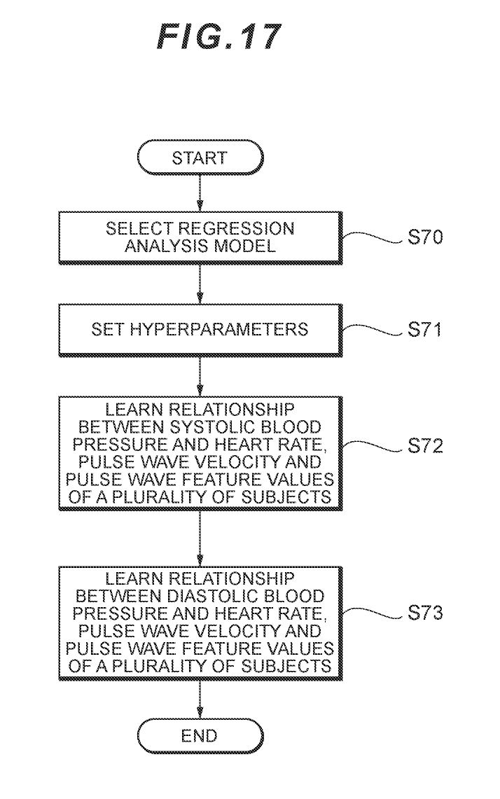

FIG. 17 is a flowchart illustrating a learning process for training the learner 17 of the blood pressure measuring device 1 according to an embodiment of the present invention. The process of training the learner 17 need not be carried out by the blood pressure measuring device 1, and may be executed by another device. The learner 17 having been trained as a result of the learning process illustrated in the figure is installed in the blood pressure measuring device 1.

In the process of training the learner 17 there is firstly selected a regression analysis model that is to be used by the learner 17 (S70). The regression analysis model may be selected from among K-nearest neighbor regression R1, random forest regression R2 and support vector regression R3. Next, hyperparameters are set for the selected regression analysis model (S71). Thereafter the first learner 17a in the learner 17 learns a relationship between systolic blood pressure and the heart rate, the pulse wave velocity and feature values pertaining to a pulse wave, of a plurality of subjects (S72). Thereafter the second learner 17b in the learner 17 learns the relationship between diastolic blood pressure and the heart rate, pulse wave velocity and feature values pertaining to a pulse wave, of the plurality of subjects (S73). The process of training the learner 17 is thereby over.

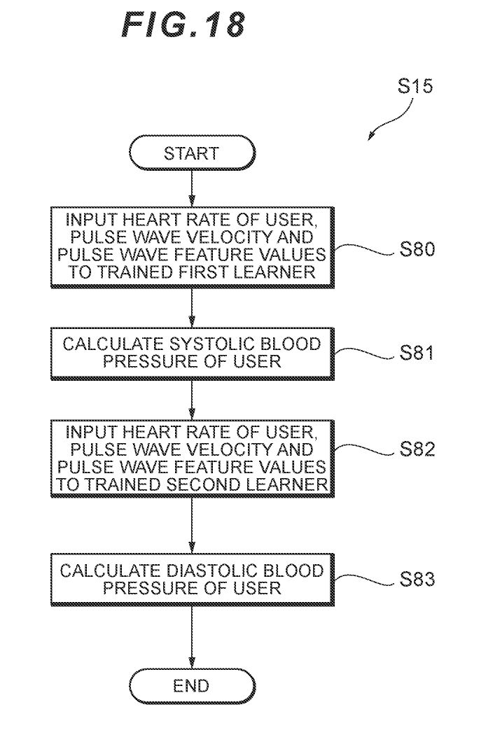

FIG. 18 is a flowchart illustrating a blood pressure calculation process by the blood pressure measuring device 1 according to an embodiment of the present invention. FIG. 18 is a flowchart illustrating the details of the blood pressure calculation process (S15) depicted in FIG. 4. Firstly, the heart rate, pulse wave velocity and feature values pertaining to a pulse wave of the user are inputted to the trained first learner 17a (S80). The systolic blood pressure of the user is then calculated by the first learner 17a (S81). Through calculation of the systolic blood pressure of the user by the first learner 17a having been trained by nonparametric regression analysis, the blood pressure measuring device 1 according to the present embodiment can calculate systolic blood pressure properly even in cases where the user is not in a resting state and modeling of a relationship between systolic blood pressure and pulse wave velocity and so forth is difficult.

The heart rate, pulse wave velocity and feature values pertaining to the pulse wave are further inputted to the trained second learner 17b (S82). The diastolic blood pressure of the user is then calculated by the second learner 17b (S83). Through calculation of the diastolic blood pressure of the user by the second learner 17b having been trained by nonparametric regression analysis, the blood pressure measuring device 1 according to the present embodiment can calculate diastolic blood pressure properly even in cases where the user is not in a resting state and modeling of a relationship between diastolic blood pressure and pulse wave velocity and so forth is difficult. It is particularly difficult to construct a physical model that expresses the relationship between diastolic blood pressure and pulse wave velocity and so forth. However, the second learner 17b allows avoiding the difficulties involved in modeling a relationship between diastolic blood pressure and heart rate, pulse wave velocity and feature values of a pulse wave, without the need for assuming a physical model that expresses such a relationship.

The purpose of the embodiments explained above is to make the present invention easy to understand, and the embodiments should not be construed as limiting the present invention in any way. For example, the arrangement, materials, conditions, shapes, sizes and so forth of the elements in the embodiments are not limited to those illustrated above, and may be modified as appropriate. Moreover, configurations in different embodiments can substitute partially for each other, or be combined with each other.

What is claimed is:

1. A blood pressure measuring device, comprising:
an electrocardiogram acquisition unit that acquires an electrocardiogram of a user;
a pulse wave acquisition unit that acquires a pulse wave of the user;
a first extraction unit that extracts a heart rate on the basis of the electrocardiogram;
a second extraction unit that extracts a pulse wave velocity on the basis of the electrocardiogram and the pulse wave;
a third extraction unit that extracts one or a plurality of feature values pertaining to the pulse wave, on the basis of the pulse wave; and
a calculation unit that calculates blood pressure of the user from the heart rate, the pulse wave velocity and the one or plurality of feature values extracted for the user, by a learner that has learned, by nonparametric regression analysis, a relationship between blood pressure of each of a plurality of subjects and a heart rate, a pulse wave velocity and one ora plurality of feature values pertaining to a pulse wave extracted for each of the plurality of subjects.

2. The blood pressure measuring device according to claim 1,
wherein the learner includes a first learner that has learned a relationship between systolic blood pressure of each of the plurality of subjects and a heart rate, a pulse wave velocity and one or a plurality of feature values pertaining to a pulse wave extracted for each of the plurality of subjects; and
the calculation unit calculates, by way of the first learner, systolic blood pressure of the user from the heart rate, the pulse wave velocity and the one or plurality of feature values extracted for the user.

3. The blood pressure measuring device according to claim 1,
wherein the learner includes a second learner that has learned a relationship between diastolic blood pressure of each of the plurality of subjects and a heart rate, a pulse wave velocity and one or a plurality of feature values pertaining to a pulse wave extracted for each of the plurality of subjects; and
the calculation unit calculates, by way of the second learner, diastolic blood pressure of the user from the heart rate, the pulse wave velocity and the one or plurality of feature values extracted for the user.

4. The blood pressure measuring device according to claim 1,
wherein the first extraction unit detects R-wave peak points of the electrocardiogram, performs an outlier exclusion process on the R-wave peak points, and thereafter extracts the heart rate on the basis of a time interval of the R-wave peak points.

5. The blood pressure measuring device according to claim 4,
wherein the second extraction unit detects a rising point of the pulse wave, a point of maximal slope of the pulse wave and a systolic peak point of the pulse wave, performs an outlier exclusion process on the rising point of the pulse wave, the point of maximal slope of the pulse wave and the systolic peak point of the pulse wave, and thereafter extracts the pulse wave velocity on the basis of a time interval of the R-wave peak point and any one of the rising point of the pulse wave, the point of maximal slope of the pulse wave and the systolic peak point of the pulse wave.

6. The blood pressure measuring device according to claim 5,
wherein the third extraction unit detects a diastolic peak point of the pulse wave, performs an outlier exclusion process on the diastolic peak point of the pulse wave, and thereafter extracts a crest ratio of the systolic peak point and the diastolic peak point of the pulse wave, a time interval of the systolic peak point and the diastolic peak point of the pulse wave, and a time interval of the rising point and the systolic peak point of the pulse wave.

7. The blood pressure measuring device according to claim 5,
wherein the third extraction unit detects a plurality of peak points of an acceleration pulse wave being the acceleration of the pulse wave, performs an outlier exclusion process on the plurality of peak points of the acceleration pulse wave being the acceleration of the pulse wave, and thereafter extracts crest ratios and time intervals between the plurality of peak points of the acceleration pulse wave.

8. The blood pressure measuring device according to claim 1,
wherein the third extraction unit compares a velocity pulse wave at rest, being the velocity of a pulse wave acquired when the user is at rest, and a velocity pulse wave during exercise, being the velocity of a pulse wave acquired when the user is exercising, performs an outlier exclusion process on the pulse wave acquired when the user is exercising, and thereafter extracts one or a plurality of feature values pertaining to the pulse wave acquired when the user is exercising.

9. The blood pressure measuring device according to claim 1, wherein the learner learns a relationship between the blood pressure of each of the plurality of subjects and a heart rate, a pulse wave velocity and one or a plurality of feature values pertaining to a pulse wave extracted for each of the plurality of subjects, by using any one of:
K-nearest neighbor regression of using blood pressure of K (K is a natural number equal to or greater than 1) subjects selected in ascending order of distance between the heart rate, the pulse wave velocity and the one or plurality of feature values extracted for the user, and a heart rate, a pulse wave velocity and one or a plurality of feature values pertaining to a pulse wave extracted for each of the plurality of subjects;
random forest regression of constructing a plurality of decision trees for deciding blood pressure on the basis of quantities randomly selected from among a heart rate, a pulse wave velocity and one or a plurality of feature values pertaining to a pulse wave extracted for each of the plurality of subjects, and using blood pressure obtained in accordance with the plurality of decision trees on the basis of the heart rate, the pulse wave velocity and the one or plurality of feature values extracted for the user; and
support vector regression of using values of a kernel function obtained on the basis of a support vector belonging to a regression hyperplane worked out so that respective margins for a heart rate, a pulse wave velocity and one or a plurality of feature values pertaining to a pulse wave extracted for each of the plurality of subjects are maximized, and on the basis of the heart rate, the pulse wave velocity and the one or plurality of feature values extracted for the user.

10. A method of measuring blood pressure, which comprises:
acquiring an electrocardiogram of a user;
acquiring a pulse wave of the user;
extracting a heart rate on the basis of the electrocardiogram;
extracting a pulse wave velocity on the basis of the electrocardiogram and the pulse wave;
extracting one or a plurality of feature values pertaining to the pulse wave, on the basis of the pulse wave; and
calculating blood pressure of the user from the heart rate, the pulse wave velocity and the one or plurality of feature values extracted for the user, by a learner that has learned, by nonparametric regression analysis, a relationship between blood pressure of each of a plurality of subjects and a heart rate, a pulse wave velocity and one or a plurality of feature values pertaining to a pulse wave extracted for each of the plurality of subjects.

11. A non-transitory computer readable medium storing a blood pressure measurement program for causing a computer to function as:
an electrocardiogram acquisition unit that acquires an electrocardiogram of a user;
a pulse wave acquisition unit that acquires a pulse wave of the user;
a first extraction unit that extracts a heart rate on the basis of the electrocardiogram;
a second extraction unit that extracts a pulse wave velocity on the basis of the electrocardiogram and the pulse wave;
a third extraction unit that extracts one or a plurality of feature values pertaining to the pulse wave, on the basis of the pulse wave; and
a calculation unit that calculates blood pressure of the user from the heart rate, the pulse wave velocity and the one or plurality of feature values extracted for the user, by a learner that has learned, by nonparametric regression analysis, a relationship between blood pressure of each of a plurality of subjects and a heart rate, a pulse wave velocity and one ora plurality of feature values pertaining to a pulse wave extracted for each of the plurality of subjects.
